(19) United States
(12) Patent Application Publication
(10) Pub. No.: US 2015/0323516 A1
(43) Pub. Date: Nov. 12, 2015

(54) Method And System For Spatially Resolved Geochemical Characterisation

(71) Applicant: Ingrain, Inc., Houston, TX (US)

(72) Inventor: Kathryn Elizabeth Washburn, Houston, TX (US)

(73) Assignee: Ingrain, Inc., Houston, TX (US)

(21) Appl. No.: 14/704,997

(22) Filed: May 6, 2015

Related U.S. Application Data
(60) Provisional application No. 61/989,621, filed on May 7, 2014.

(51) Int. Cl. G01N 33/24

(52) U.S. Cl. CPC G01N 33/241

(57) ABSTRACT

A method which allows for determining geochemistry with spatial resolution of geological materials or other materials is provided. The method can provide a non-bulk method of characterizing the geochemistry of a sample with spatial resolution. A system for performing the method also is provided.

This application claims the benefit under 35 U.S.C. §119(e) of prior U.S. Provisional Patent Application No. 61/989,621, filed May 7, 2014, which is incorporated in its entirety by reference herein.

### FIELD OF THE INVENTION

The present invention relates to spatially resolved geochemical characterisation and, more particularly, to a method for determining geochemistry with spatial resolution, and a system for making such determinations, which can be used for determining geochemistry of geological materials, such as rocks, or other materials.

### BACKGROUND OF THE INVENTION

Characterisation of source rocks is important for evaluation of both conventional and unconventional reservoirs. Organic matter is deposited and preserved at the bottom of lakes, seas and deltas. As more material is deposited, the organic matter is buried and the heat and pressure of burial transforms the organic matter into geopolymers such as kerogen and bitumen. When the rocks containing organic matter are buried deep enough, the rocks undergo catagenesis where temperature begins to convert the kerogen into bitumen and ultimately into hydrocarbons such as oil and gas. The rocks that produce hydrocarbons are referred to as source rocks.

Kerogen and bitumen are large organic molecules of no fixed structure. The composition of the matter depends both on the type of organic matter used to produce the geopolymers and the thermal maturity of the sample. While kerogen and bitumen have different molecular structures, they are typically separated functionally; the latter is soluble in common organic solvents while the former is not. The majority of bitumen is produced during catagenesis, though a small amount occurs from diagenesis.

Understanding kerogen and bitumen is important for estimation of thermal maturity and potential hydrocarbon production. Thermal maturity indicates how much and what type of hydrocarbon is expected to have been produced by the source rock. In addition to kerogen and bitumen, a third class of organic matter, pyrobitumen, may exist in more thermally mature systems. Like kerogen, pyrobitumen is also insoluble in typical organic solvents. However, while kerogen originates from the originally deposited organic matter, the pyrobitumen comes from the cracking of bitumen during catagenesis and metagenesis.

The current standard method for determining thermal maturity is programmed pyrolysis, such as the “Rock-Eval™” or “Source Rock Analysis” techniques. These systems will heat up a crushed portion of sample to a given temperature. The sample is held at an initial temperature for a period of time and the produced organic compound products are measured using a flame ion detector (FID). This is referred to as the S1 peak, which relates to the free hydrocarbon and bitumen content in the sample. The temperature is then ramped higher and again held for a period of time, where the produced organic compounds are measured again by FID. The produced organic compounds at this temperature are associated with volitisation of kerogen and are referred to as the S2 peak. As the sample cools, there is a release of carbon dioxide (CO₂) and carbon monoxide (CO) that is measured by infrared detectors. This peak, S3, is associated with the organic associated oxygen in the sample. There is the potential to heat the sample up to even higher temperatures and observe the produced products. The high temperature programmed pyrolysis is used to measure the pyrobitumen identified in spent shale (S_{py} peak).

The programmed pyrolysis methods are bulk methods; the samples need to be crushed and homogenized before measurement. Therefore, any spatial information regarding the distribution of organic matter is lost during the crushing process. They are also destructive, as the samples cannot be used for further tests after programmed pyrolysis. Programmed pyrolysis measurements are time intensive, usually requiring about an hour per sample to perform. The results also can have issues with interference from carbonate in the sample. If the samples are carbonate rich, they will need to be pretreated with hydrochloric acid to prevent interference in the measurement.

Thermal maturity is often estimated using the temperature where the maximum number of organic compound products are produced. This can be unreliable, as the peaks are often quite broad, such the exact location of the peak can vary and can be difficult to reproduce with subsequent measurements.

Fourier Transform Infrared (FTIR) spectroscopy has been used to estimate these geochemical parameters. Analysis of the FTIR spectrum with multivariate analysis has shown good predictive value for geochemical parameters such as S1, S2, and to a lesser degree S3. Predictive ability of FTIR to date for hydrogen and oxygen indices have been of poor quality. FTIR suffers the same drawback of loss of spatial resolution of the organic matter as the programmed pyrolysis, as samples are often powdered before measurement.

### SUMMARY OF THE INVENTION

A feature of the present invention is a method for determining geochemistry with spatial resolution for geological materials such as rock samples or other materials.

A further feature of the present invention is a system for making such determinations.

To achieve these and other advantages and in accordance with the purposes of the present invention, as embodied and broadly described herein, the present invention relates, in part, to a method for determining geochemistry of at least one sample, comprising a) obtaining spectral data on the at least one sample, b) obtaining spatial information on at least one sample, c) obtaining geochemical information on the at least one sample using the spectral data, and d) determining spatially resolved geochemical information for the at least one sample using the geochemical information and the spatial information.

A system for performing the method is also provided.

The present invention further relates to a method for determining geochemical information relating to kinetic analysis of a sample, comprising: a) heating at least one sample by laser-induced pyrolysis, such as LIBS; b) monitoring the reaction rate, such as a value of the Arrhenius equation rate constant k, of at least one sample comprising at least one of: i) monitoring changes in amounts of elements associated with organic matter and hydrocarbons for a portion of at least one sample that is heated by the laser-induced pyrolysis, ii) collecting and analysing hydrocarbon species produced by pyrolysis of a portion of at least one sample from the laser-induced pyrolysis by a flame ion detector or gas chromatography-mass spectrometry (GC-MS), iii) monitoring the weight of at least one sample during the laser-induced pyrolysis of at least one sample, iv) monitoring the temperature of at least one sample and determining the amount of energy inputted into the portion of the sample by the laser during the laser-induced pyrolysis. The prefactor in the Arrhenius equation may be inputted based on a priori knowledge or solved for based on measurements performed on two or more different heating rates of the sample. The different heating rates may be obtained by one or more combinations of different laser power, laser spot size or laser shot rate. The kinetic analysis by LIBS can be used to either solve for the activation energy distribution in the sample or the reaction rates given a known input of energy (e.g., inputted laser energy).

It is to be understood that both the foregoing general description and the following detailed description are exemplary and explanatory only and are intended to provide a further explanation of the present invention, as claimed.

The accompanying FIGURES, which are incorporated in and constitute a part of this application, illustrate various features of the present invention and, together with the description, serve to explain the principles of the present invention. The features depicted in the figures are not necessarily drawn to scale. Similarly numbered elements in different FIGURES represent similar components unless indicated otherwise.

### BRIEF DESCRIPTION OF THE DRAWINGS

FIG. 1 shows a process flow chart of the determining of spatially resolved geochemistry of a sample according to an example of the present application.

### DETAILED DESCRIPTION OF THE INVENTION

The present invention relates in part to a method which allows for determining geochemistry with spatial resolution of rocks or other materials. Further, the method can provide a non-bulk method for characterizing the geochemistry of a sample with spatial resolution. The method can be practiced as a rapid, non-destructive geochemical analysis method with respect to a sample. The measurements can be performed on the exact same samples or different samples of similar composition and structure can be used to estimate geochemistry information that does not require preparation. The results of the method of this invention may be used to distinguish kerogen and bitumen in the samples. Rapid thermal maturity estimates can be translated along the length of a core. Spatially resolved maps obtained with the method of the present invention can be applied to sample models to help distinguish between kerogen and bitumen in the models.

The materials, also referred to herein as the samples, to which the present invention can be applied are not necessarily limited. The materials can be geological materials, such as rocks or samples thereof. The kinds of rock to which a method of the present invention can be applied are not necessarily limited. The rock sample can be, for example, organic mud rock, shale, carbonate, sandstone, limestone, dolostone, or other rocks, or any combinations thereof, or other kinds. The rocks can be porous or non-porous. Any source of a rock formation sample of manageable physical size and shape may be used with the present invention. Micro-cores, crushed or broken core pieces, drill cuttings, sidewall cores, outcrop quarrying, whole intact rocks, and the like, may provide suitable rock piece or fragment samples for analysis using methods according to the invention.

The present invention relates in part to a method for determining geochemistry of a sample that includes steps of obtaining spectral data on a sample, obtaining spatial information on the sample, obtaining geochemical information on the sample using the spectral data, and determining spatially resolved geochemical information for the sample using the geochemical information and spatial information. Spectral and spatial measurements may be performed on the exact same sample, or two or more samples of similar composition and structure.

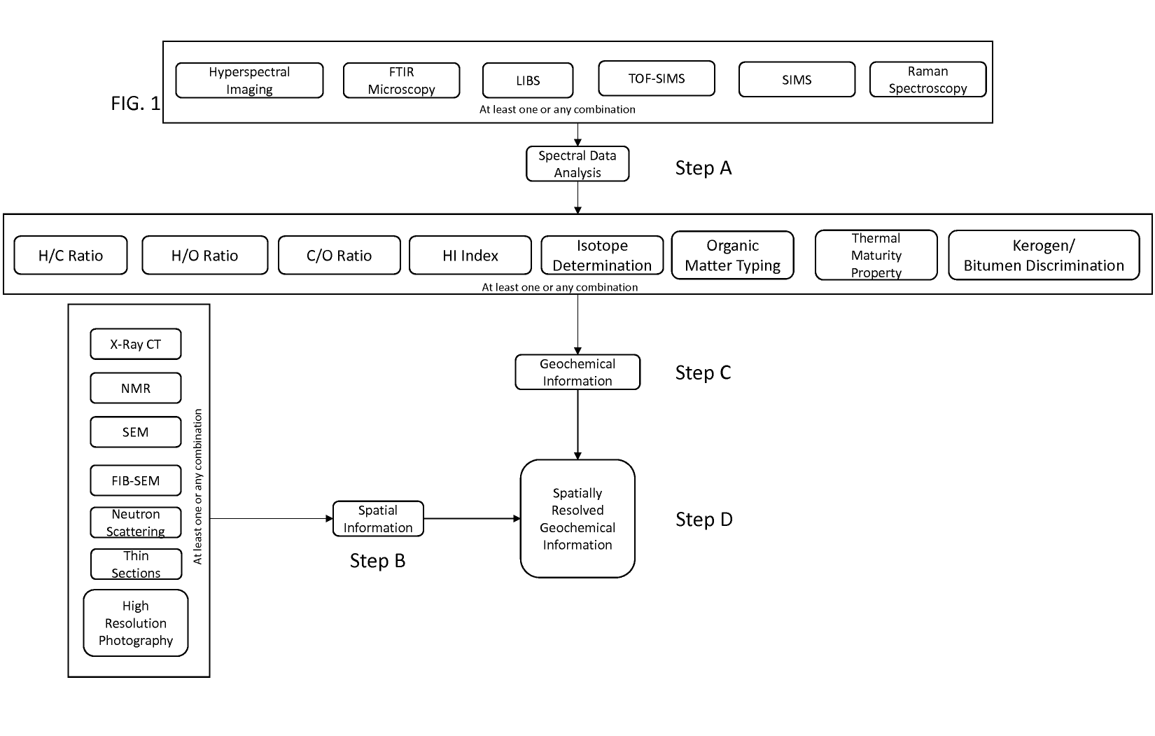

Referring to FIG. 1, a process flow of a method of the present invention is illustrated which includes Steps A, B, C, and D.

The spectral measurement focus can be on organic matter, inorganic matter, or both organic and inorganic matter, and the contributions of the organic matter and inorganic matter can be deconvoluted through manual identification, univariate or multivariate analysis.

In Step A, spectral data is obtained. The spectra are generated by, but not limited to, LIBS, TOF-SIMS, SIMS, FTIR, FTIR microscopy, Raman spectroscopy or Hyperspectral Imaging, or other equipment capable of generating spectral data. The spectra data can be used to create geochemical information about the surface of the sample.

In Step B, spatial information/data is obtained. Spatial information can be generated by, but not limited to, X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or other equipment capable of generating spatial information.

The samples can undergo spectral measurement and spatial imaging in the same setup, or the samples can undergo spectral measurement and then are transferred to a second setup for spatial imaging, or the samples can undergo spatial imaging and are then transferred to a second equipment for spectral measurement, or the samples can undergo spectral measurement and spatial imaging and one or more intermediate measurements between the two types of measurements. Spectral and spatial measurements may be performed on the exact same sample or the spectral measurement can be performed on one sample(s) and the spatial measurement performed on a second sample(s) where samples are of similar composition and structure.

In Step C, the spectra is correlated to provide geochemical information. The correlation in Step C can comprise one or more of the following:

a) univariate analysis is used to correlate the spectra to atomic hydrogen/carbon (H/C) ratio, or
b) multivariate analysis is used to correlate the spectra to H/C ratio, or
c) univariate analysis is used to correlate the spectra to atomic hydrogen/oxygen (H/O) ratio, or
d) multivariate analysis is used to correlate the spectra to H/O ratio, or
e) univariate analysis is used to correlate the spectra to atomic carbon/oxygen (C/O) ratio, or
f) multivariate analysis is used to correlate the spectra to C/O ratio, or
g) univariate analysis is used to correlate the spectra to hydrogen index, or
h) multivariate analysis is used to correlate the spectra to hydrogen index, or
i) univariate analysis is used to correlate the spectra to oxygen index, or
j) multivariate analysis is used to correlate the spectra to oxygen index, or
k) univariate analysis is used to correlate the spectra to the results from programmed pyrolysis, or
l) multivariate analysis is used to correlate the spectra to the results from programmed pyrolysis, or

m) univariate analysis is used to correlate the spectra to a thermal maturity property (e.g., thermal maturity, kinetic analysis), or

n) multivariate analysis is used to correlate the spectra to a thermal maturity property (e.g., thermal maturity, kinetic analysis), or
o) univariate analysis is used to correlate the spectra to kerogen and bitumen content, or
p) multivariate analysis is used to correlate the spectra to kerogen and bitumen content, or
q) univariate analysis is used to correlate the spectra to kerogen type, or
r) multivariate analysis is used to correlate the spectra to kerogen type, or
s) univariate analysis is used to correlate the spectra to hydrocarbon content, or
t) multivariate analysis is used to correlate the spectra to hydrocarbon content, or
u) univariate analysis is used to correlate the spectra to hydrocarbon type, or
v) multivariate analysis is used to correlate the spectra to hydrocarbon type, or
w) multivariate analysis is used to correlate the spectra to isotope analysis, or
x) univariate analysis is used to correlate spectra to isotope analysis.

Any single one or any combination of two or more, or three or more, or four or more and so forth, of the correlations in a)-x) can be used in Step C.

In Step D, the spectral data is integrated into two or three dimensional models created from spatial imaging, to generate spatially resolved geochemical information on the sample. Appropriate spatial geochemistry information in the 2D or 3D models can be determined through image segmentation, assigned manually, determined by capillary pressure simulation or measurements, or determined from previously spatially resolved spectral measurements.

As indicated, spectral information that can be used to assess geochemistry of the samples in methods of the present invention can be obtained by a variety of methods including, but not limited to, FTIR, FTIR microscopy, SIMS, TOF-SIMS, LIBS, Raman spectroscopy and Hyperspectral Imaging. FIG. 1 shows many of these modes of spectral data acquisition, which can have the following features and/or others.

Laser induced breakdown spectroscopy (LIBS) uses a laser to ablate a tiny portion of sample. The standard for LIBS uses a q-switched solid state laser that produces a rapid pulse, typically on the order of pico- to nanoseconds in duration. Optics are used to focus the energy onto a single spot on the sample. The laser ablates a small amount of sample at this spot, turning it into a high temperature plasma. The excited atoms then return to a ground state, giving off light of characteristic frequencies. The spot size vaporized by the laser can range in size from a few microns up to hundreds of microns, allowing a large range of resolution and is dependent on the optics of the system. The signal quality improves with larger spot size, but sacrifices resolution. While a small amount of sample is consumed, the amount is so small that it is considered to be negligible and the technique is considered non-destructive. The wavelength of light from the plasma is in the 200 to 980 nm region. The resulting spectra can be analysed by multivariate data to correlate the spectra to concentration of elements. LIBS has been used previously as a method for mineralogy identification, making it an alternative to X-ray Diffraction (XRD) and X-ray Fluorescence (XRF) methods for mineralogical analysis of samples. It has an advantage over XRF for mineralogical identification because it can measure all elements, whereas XRF is unable to detect light elements. LIBS is able to perform depth profiling, firing the laser in the same spot and observing the different products that are produced with increased depth. LIBS is also very rapid, only taking seconds per measurement making it amenable for high-throughput industrial use. LIBS measurements can be rastered to produce a two dimensional map of surface composition.

As another type of geochemical information that can be obtained, laser-induced pyrolysis, e.g., LIBS, which can be used to perform a rapid kinetic analysis for determining how thermal maturation of one or more samples progresses depending on the energy input. For purposes herein, a reaction rate can refer to a generation rate for hydrocarbons from thermally-induced decomposition of kerogen in the sample, e.g., a hydrocarbon generation rate. In evaluating generation rates using kinetics analysis, the quantity, types, and rate at which hydrocarbons are generated from kerogen given particular heating conditions can be estimated in addition to determining what type and quantity of hydrocarbons the kerogen may already have produced. Kinetic analysis can be used to help understand the conversion process of organic matter from kerogen into products like thermobitumen, oil, gas, and pyrobitumen. This can be used to help understand what petroleum products may have been produced by source rocks and reservoir rocks, such as for the case of tight oil and gas shales, and for the case of oil shale, what petroleum products may be produced in the future, and at what generation rates. Kerogen maturation can be considered to be tied to chemical reaction rates. Many kinetic formulations assume that kerogen directly converts to oil and gas hydrocarbons, or other formulations assume that kerogen converts to hydrocarbons via bitumen intermediate. Kinetic models can use the Arrhenius equation, which is given by equation (1): k=Ae^{-Ea/RT}. In the indicated Arrhenius equation, k is the rate constant of the chemical reaction, such as the reaction rate constant for loss of the reacting (decomposing) species of kerogen in the transformation of kerogen to hydrocarbons, which can be expressed as the change in the molar mass of the reactant with respect to time. A is the pre-exponential or frequency factor, which describes the number of potential elementary reactions per unit time (e.g., in units of min¹). Eₐ is the activation energy that describes the energy barrier that must be exceeded in order for a reaction to occur (in energy/mole, e.g., kiloJoule/mole). R is the gas constant (e.g., 0.008314 kJ/° K-mole), and T is the absolute temperature (° K). If kinetic analysis is performed by running programmed pyrolysis measurements, the temperature of the oven is known, the quantity of produced organic products monitored and can be used to obtain the distribution of Eₐ value for a sample. When determining Eₐ from data obtained using a pyrolysis oven in programmed pyrolysis measurements, a challenge is in determining the value of A. Typically several programmed pyrolysis measurements can be performed with different heating rates for purposes of solving for the value of A. In kinetic analysis that uses a multiple-heating ramp open-system pyrolysis strategy, kinetic analyses begins with pyrolysis of source rock samples in an oven using two, three, or more different heating rates (e.g., different ° C./min heating rates). When the reaction in question is first order and occurs under isothermal conditions, then activation energies (Eₐ) and frequency factors (A) may be obtained from a plot of the natural logarithm of the reaction rate (ln k) versus the inverse of the absolute temperature (1/T), where k is the reaction rate (mass/time) and T is the temperature (T in ° K). Activation energies and frequency factors also may be found using non-isothermal experiments as long as the temperature varies at a constant rate. An approximate solution for the Arrhenius equation under those conditions can use the Kissinger method or other approaches. E.g., S. H. Nordeng, “Evaluating Source Rock Maturity Using Multi-Sample Kinetic Parameters . . . ,” Geol. Investig. No. 164, North Dak. Geol. Survey, 2013, pp. 1-19, incorporated in its entirety by reference herein. In some cases, A can be either fixed or assigned from a priori knowledge such that only one heating rate is necessary in a one-run, open-system pyrolysis experiment (“single ramp” pyrolysis). The present invention can include a method for determining kinetic properties, such as reaction rates or activation energies for a sample that does not require heating of an entire sample in a pyrolysis oven and can provide reliable information on how a sample has and will thermally mature.

Instead of heating an entire sample in an oven to generate data for kinetic modeling, in the present invention, a laser can be used to pyrolyse the sample at a single or multiple selected locations, such as discrete spots on the sample. Data can be acquired from this method using laser-induced pyrolysis that can be used in a kinetic analysis of the sample. The generated data can be locationally-mapped across a surface of the sample, and/or for different depths of the same sample (or different sample). A laser can be used as the source of heat that pyrolyzes the sample, and k, Eₐ and/or other kinetic property data can be determined for the laser-heated portion of the sample by one or several different strategies. In this respect, k, Eₐ and/or other kinetic property data can be determined from data obtained during laser heating of a portion of the sample based on changes in amounts of elements associated with organic matter and hydrocarbons, e.g., by monitoring the increase or decrease in elements associated with organic matter and hydrocarbons. In another respect, k, Eₐ and/or other kinetic property data can be determined from data obtained during laser heating of a portion of the sample by collection and analysis of the produced hydrocarbon species by a flame ion detector or gas chromatography-mass spectrometry (GC-MS), or by monitoring the weight of the sample during the laser-induced pyrolysis. Alternatively, as the amount of energy inputted into the system by the laser is known, by monitoring the temperature of the sample, k can be calculated for a portion of the sample that is heated by the laser-induced pyrolysis. In these respects, a single LIBS measurement can be performed, or multiple measurements can be performed which can have the same or different settings of the laser power, repetition rate, or spot size. A LIBS measurement can comprise one of more shots of a laser followed by the observation of the emitted spectra. Temperature can be assumed based on prior information, or calculated through the intensity of the LIBS peaks in the spectra, or by monitoring the sample through a device such as an infrared (IR) camera. A combination of monitoring the inputted energy to the system, the sample temperature, and produced products can provide an understanding of the chemical kinetics of the organic matter maturation, such as the reaction rate or distribution of activation energies. If an IR camera is used in determining the sample temperature resulting from the laser treatment, in addition to understanding the kinetics analysis of the organic matter, the heat transfer properties of the shale can be observed by monitoring the temperature of the sample after laser shots and how the temperature changes around the laser spot as a function of time.

Time of Flight Secondary Ion Mass Spectroscopy (TOF-SIMS) uses ions to dislodge molecules from sample surfaces. A variety of ions can be used, including but not limited to Ga, Au, Au2, Au3, Bi, Cs, and C60 ions. The ions can be used with energies which can range from about 0.3 to about 30 keV, such as from about 1 to about 25 keV or from about 1 to about 10 keV, or other range values. Unlike dynamic SIMS, lower energies are used such that molecular structure of the ablated material remains intact. In dynamic SIM, higher energy is used such that the molecular structure is broken and only elements are measured. The ablated components for TOF-SIMS are then accelerated to a constant kinetic energy. If kinetic energy is held constant, then the time the species take to travel will vary depending on their mass. By measuring the time of flight, the time it takes for the molecular species to travel though the detector, their mass can be determined. From component mass, the molecular species can then be identified. The measurements are performed as a raster, such that a high resolution map of surface composition can be created. Results have then been analysed using multivariate analysis techniques, such as principle component analysis and partial least squares regression to relate surface composition. TOF-SIMS has been used to determine contact angle for a variety of different industries such as semi-conductors, medical industry. The mining industry has used TOF-SIMS to determine surface wettability of geology samples to estimate how well different components will separate during floatation separation.

Dynamics Secondary Mass Spectroscopy uses ions to dislodge molecules from sample surfaces. A variety of ions can be used, including, but not limited to, Ar, Xe, O, SF5 and C60. A mass spectrometer is then used to measure the mass of the produced species. The energy of the ions used is such that the molecular bonds of the surface materials are broken and only the elements are measured. The measurements are performed as a raster, such that a high resolution map of surface composition can be created. Results have then been analysed using multivariate analysis techniques, such as principle component analysis and partial least squares regression to relate surface composition.

Fourier transform infrared (FTIR) microscopy combines FTIR measurements with spatial resolution to produce a FTIR spectrum. FTIR works by shining infrared light upon a sample. Depending on the composition of the sample, some wavelengths of light will be absorbed while others will pass through the sample. The transmitted light is then measured to produce a spectra showing absorption profile as a function of wavelength. Organic matter and inorganic minerals have characteristic absorption profiles which can be used to identify sample constituents. This may be done qualitatively or quantitatively by use of mineral libraries, manual identification, univariate analysis or multivariate analysis. The FTIR microscope advances normal FTIR measurements by combining the technique with an optical microscope such that individual areas of a sample can be selected and FTIR spectra taken, allowing composition at a higher resolution to be determined. Unlike standard FTIR measurements which are normally performed on powders, the FTIR microscopy can be performed on intact samples. Standard procedure for geological FTIR microscopy uses a sample that is polished to produce an even surface. FTIR microscopy can be performed via transmission FTIR, diffuse reflectance infrared Fourier transform spectroscopy (DRIFTS) or attenuated total reflectance (ATR) FTIR.

Raman spectroscopy uses monochromatic light, usually from a laser, to excite rotational and vibrational modes in a sample. Raman spectroscopy measures the Raman scattering, the inelastic scattering that occurs when light interacts with matter. When photons from the laser interact with the molecular vibrations in the sample, they change the excitation state of the molecule. As the molecule returns to equilibrium, this results in the emission of an inelastically scattered photon that may be of higher or lower frequency than the excitation depending on whether the final vibration state of the molecule is higher or lower than the original state. These shifts give information on the vibrational and rotational modes of the sample, which can be related to its material composition. The signal to noise of Raman spectroscopy tends to be weaker compared to other methods such as FTIR.

Hyperspectral imaging creates a spectra for each pixel of an image. Light from an object passes through a dispersing element, such as a prism or a diffraction grating, and then travels to a detector. Optics are typically used in between the dispersing element and the detector to improve image quality and resolution. Hyperspectral imaging may range over a wide range of light wavelengths, including both visible and non-visible light. Multispectral is a subset of hyperspectral imaging that focuses on a few wavelengths of key interest. Hyperspectral imaging is defined by measuring narrow, well defined contiguous wavelengths. Multispectral imaging instead has broad resolution or the wavelengths to be measured are not adjacent to each other. Hyperspectral imaging has been used previously in a wide range of industries. In particular, hyperspectral imaging has been used in aerial mounted surveys to determine mineralogy for oil, gas, and mineral exploration.

FIG. 1 also shows modes of spatial information acquisition, including X-ray CT, NMR, SEM, FIB-SEM, neutron scattering, thin sections and high resolution photography. These can be adapted for use in the present invention from known equipment and manners of use.

The present invention includes the following aspects/embodiments/features in any order and/or in any combination:

1. The present invention relates to a method for determining geochemistry of a sample, comprising:
a) obtaining spectral data on at least one sample;
b) obtaining spatial information on at least one sample;
c) obtaining geochemical information on the at least one sample using the spectral data;
d) determining spatially resolved geochemical information for the at least one sample using the geochemical information and the spatial information, wherein the sample in a) and the sample in b) are the same or are different but have the same or similar composition and structure.
2. The method of any preceding or following embodiment/feature/aspect, wherein the spectral data on the sample is generated by LIBS, TOF-SIMS, SIMS, FTIR, FTIR microscopy, Raman spectroscopy, hyperspectral imaging, or any combinations thereof.
3. The method of any preceding or following embodiment/feature/aspect, wherein the spatial information on the sample is obtained by X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or any combinations thereof.
4. The method of any preceding or following embodiment/feature/aspect, wherein the sample undergoes spectral measurement and spatial imaging in the same setup, or the sample undergoes spectral measurement and then is transferred to a second setup for spatial imaging, or the sample undergoes spatial imaging and is then transferred to a second equipment for spectral measurement, or the sample undergoes spectral measurement and spatial imaging and one or more intermediate measurements between the two types of measurements. Spectral and spatial measurements may be performed on the exact same sample, or two or more samples of similar composition and structure.
5. The method of any preceding or following embodiment/feature/aspect, wherein the geochemical information is obtained with determined values for H/C ratio, H/O ratio, CIO ratio, HI index, OI index, isotope determination, organic matter typing, thermal maturity, kerogen/bitumen discrimination, or any combinations thereof.
6. The method of any preceding or following embodiment/feature/aspect, wherein the spatially resolved geochemical information is provided to a 2D or 3D model that is determined through image segmentation, assigned manually, determined by capillary pressure simulation or measurements, or determined from previously spatially resolved spectral measurements.
7. The method of any preceding or following embodiment/feature/aspect, wherein the sample is a geological sample.
8. The method of any preceding or following embodiment/feature/aspect, wherein the sample is a rock sample.
9. The present invention further relates to a method for determining geochemistry of a sample, comprising:
a) obtaining spectral data on at least one sample, wherein the spectral data on the sample is generated by laser-induced pyrolysis, such as LIBS;
b) obtaining spatial information on at least one sample;
c) obtaining geochemical information for at least one sample using the spectral data, wherein the geochemical information comprises kinetic analysis for at least one sample;
d) determining spatially resolved geochemical information for at least one sample using the geochemical information and the spatial information, wherein the sample in a) and the sample in b) are the same or are different but have the same or similar composition and structure.
10. The present invention further relates to a method for performing kinetic analysis as geochemical information of a sample, comprising:
a) heating at least one sample by laser-induced pyrolysis, such as LIBS;
b) determining a reaction rate, such as a value of the Arrhenius equation rate constant k, of at least one sample comprising at least one of:
i) determining changes in amounts of elements associated with organic matter and hydrocarbons for a portion of at least one sample that is heated by the laser-induced pyrolysis,
ii) collecting and analysing hydrocarbon species produced by pyrolysis of a portion of at least one sample from the laser-induced pyrolysis by a flame ion detector or gas chromatography-mass spectrometry (GC-MS),
iii) monitoring weight of at least one sample during the laser-induced pyrolysis of at least one sample,
iv) monitoring the temperature of at least one sample and determining the amount of energy inputted into the portion of the sample by the laser during the laser-induced pyrolysis, or using any combination of i), ii), iii), and iv), such as ii) and/or iii) in conjunction with either i) or iv).
11. The method of any preceding or following embodiment/feature/aspect, wherein a prefactor in the Arrhenius equation is inputted based on a priori knowledge or solved for based on measurements performed on two or more different heating rates of the sample.
12. The method of any preceding or following embodiment/feature/aspect, wherein the different heating rates are obtained by one or more of different laser power, laser spot size or laser shot rate, or any combination thereof.
13. The method of any preceding or following embodiment/feature/aspect, wherein the kinetic analysis by LIBS is used to either solve for the activation energy distribution in the sample or the reaction rates given a known input of energy.
14. A system to perform any of the methods of any preceding claim.
15. A system for determining geochemistry of a sample, comprising i) a spectral data acquisition device for obtaining spectral data on at least one sample; ii) a spatial information acquisition device for obtaining spatial information on at least one sample, wherein the spectral data acquisition device and the spatial information acquisition device are the same device or different devices, and wherein the sample used in i) and the sample used in ii) are the same or are different but have the same or similar composition and structure; iii) one or more computer systems comprising at least one processor and/or computer programs stored on a non-transitory computer-readable medium operable to obtain geochemical information on the sample used in i) using the spectral data, and to determine spatially resolved geochemical information for the sample or samples used in i) and ii) using the geochemical information and the spatial information; and iv) at least one device to display, print, and/or store as a non-transitory storage medium, results of the computations.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method for determining geochemistry of a sample, comprising:
a) obtaining spectral data on at least one sample;
b) obtaining spatial information on at least one sample;
c) obtaining geochemical information on the at least one sample using the spectral data;
d) determining spatially resolved geochemical information for the at least one sample using the geochemical information and the spatial information, wherein the sample in a) and the sample in b) are the same or are different but have the same or similar composition and structure.

2. The method of claim 1, wherein the spectral data on the sample is generated by LIBS, TOF-SIMS, SIMS, FTIR, FTIR microscopy, Raman spectroscopy, hyperspectral imaging, or any combinations thereof.

3. The method of claim 1, wherein the spatial information on the sample is obtained by X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or any combinations thereof.

4. The method of claim 1, wherein the sample undergoes spectral measurement and spatial imaging in the same setup, or the sample undergoes spectral measurement and then is transferred to a second setup for spatial imaging, or the sample undergoes spatial imaging and is then transferred to a second equipment for spectral measurement, or the sample undergoes spectral measurement and spatial imaging and one or more intermediate measurements between the two types of measurements. Spectral and spatial measurements may be performed on the exact same sample, or two or more samples of similar composition and structure.

5. The method of claim 1, wherein the geochemical information is obtained with determined values for H/C ratio, H/O ratio, C/O ratio, HI index, OI index, isotope determination, organic matter typing, thermal maturity, kerogen/bitumen discrimination, or any combinations thereof.

6. The method of claim 1, wherein the spatially resolved geochemical information is provided in a 2D or 3D model that is determined through image segmentation, assigned manually, determined by capillary pressure simulation or measurements, or determined from previously spatially resolved spectral measurements.

7. The method of claim 1, wherein the sample is a geological sample.

8. The method of claim 1, wherein the sample is a rock sample.

9. A method for determining geochemistry of a sample, comprising:
a) obtaining spectral data on at least one sample, wherein the spectral data on the sample is generated by laser-induced pyrolysis;
b) obtaining spatial information on at least one sample;
c) obtaining geochemical information for at least one sample using the spectral data, wherein the geochemical information comprises kinetic analysis for at least one sample;
d) determining spatially resolved geochemical information for at least one sample using the geochemical information and the spatial information, wherein the sample in a) and the sample in b) are the same or are different but have the same or similar composition and structure.

10. A method for performing kinetic analysis as geochemical information of a sample, comprising:
a) heating at least one sample by laser-induced pyrolysis;
b) determining a reaction rate constant k for the Arrhenius equation of at least one sample, comprising at least one of:
i) determining changes in amounts of elements associated with organic matter and hydrocarbons for a portion of at least one sample that is heated by the laser-induced pyrolysis,
ii) collecting and analysing hydrocarbon species produced by pyrolysis of a portion of at least one sample from the laser-induced pyrolysis by a flame ion detector or gas chromatography-mass spectrometry,
iii) monitoring weight of at least one sample during the laser-induced pyrolysis of the least one sample,
iv) monitoring temperature of at least one sample and determining the amount of energy inputted into the portion of the sample by the laser during the laser-induced pyrolysis.

11. The method of claim 10, wherein a prefactor in the Arrhenius equation is inputted based on a priori knowledge or solved for based on measurements performed on two or more different heating rates of the sample.

12. The method of claim 11, wherein the different heating rates are obtained by one or more of different laser power, laser spot size or laser shot rate, or any combination thereof.

13. The method of claim 10, wherein the kinetic analysis by LIBS is used to either solve for the activation energy distribution in the sample or the reaction rates given a known input of energy.

14. A system for determining geochemistry of a sample, comprising i) a spectral data acquisition device for obtaining spectral data on at least one sample; ii) a spatial information acquisition device for obtaining spatial information on at least one sample, wherein the spectral data acquisition device and the spatial information acquisition device are the same device or different devices, and wherein the sample used in i) and the sample used in ii) are the same or are different but have the same or similar composition and structure; iii) one or more computer systems comprising at least one processor and/or computer programs stored on a non-transitory computer-readable medium operable to obtain geochemical information on the sample used in i) using the spectral data, and to determine spatially resolved geochemical information for the sample or samples used in i) and ii) using the geochemical information and the spatial information; and iv) at least one device to display, print, and/or store as a non-transitory storage medium, results of the computations.
